(19) United States
(12) Patent Application Publication
(10) Pub. No.: US 2006/0155208 A1
(43) Pub. Date: Jul. 13, 2006

(54) Human body composition monitoring and measuring apparatus of a glass platform with film coating electrodes

(76) Inventors: Yuegiu Li, Shenzhen (CN); Yan Liu, Shenzhen (CN)

Correspondence Address: WESTMAN CHAMPLIN & KELLY, P.A., SUITE 1400, 900 SECOND AVENUE SOUTH, MINNEAPOLIS, MN, 55402-3319, US

(21) Appl. No.: 10/535,963

(22) PCT Filed: Jun. 19, 2003
(86) PCT No.: PCT/CN03/00476

§ 371 Date: May 24, 2005

(30) Foreign Application Priority Data
Nov. 26, 2002 (CN) 02249850.8

(51) Int. Cl. A61B 5/05

(52) U.S. Cl. 600/547

(57) ABSTRACT

The present invention relates to a measuring technology, in particular relates to a monitoring and measuring apparatus of a glass platform with film coating electrodes: a human body composition monitoring and measuring apparatus of a glass platform with film coating electrodes, comprises a glass measuring platform with film coating electrodes on the surface, a weighing sensor and a circuit, a microprocessor system, a body impedance and liquid measuring electrodes and a circuit, a display and a keyboard. The person to be measured stands on the glass measuring platform with feet bear, his soles contacting with the film coating electrodes. The parameters of body weight, body at content or water content, etc. are measured. The apparatus is convenient to be used to measure a human body composition in daily life.

### FIELD OF THE INVENTION

The present invention relates to measurement devices in general, and more particularly to a body composition monitor by measuring bioelectrical impedance based on plating electrode film on glass platform.

### BACKGROUND OF THE INVENTION

The method for estimating body composition is simple and credible by measuring bioelectrical impedance jointly using the corresponding mathematic mode. As disclosed in China patent of Electronic Weight Apparatus for Measuring Body Fat Content (ZL 00239320.4), the measuring plat is part of the scale plan, which is fixed with electrode on the surface, the body impedance can be get by the inner circuit of apparatus as the barefoot connects the electrode, then the body fat mass is estimated by the intelligent chip according to the measured body weight, body impedance, and the input object height and edge. It can get the two parameter of body weight and body fat mass.

As disclosed in China patent of Integrate Apparatus for Measuring Body Weight, Body Fat and Body Water Content (ZL 01235598.4), the measurement for main body composition parameters of weight, fat and water is integrated to the whole monitor.

The measure plat of these apparatus is fixed with metal electrodes on the plastic shell surface, however, which has the shortcomings of making plastic shell by plastic mould and of metal electrode by metal mould respectively.

### SUMMARY OF THE INVENTION

The present invention aims to providing a body composition monitor based on plating electrode film on glass platform, according to which, the parameters of body weight, fat and water is determined through the connection of foot and plating electrode as the barefoot subject stands on the glass platform, and the monitor is expediently used to monitor body composition in everyday life.

The present invention aims to providing a apparatus for monitoring body composition by plating electrode on glass platform, which includes glass platform with plating film electrode for measuring body impedance, weighing sensor, and circuit, microprocessor system, body impendence and water test electrode and circuit, display, keyboard and so on. Especially, a group of capacitance grid film electrode for measuring the dielectric constant of body endermic tissues is platted on the surface of glass platform at least, every which includes two equal interval electrode films that can't conduct each other, also with other electrodes.

The body composition monitor as mentioned on its surface there is a glass test platform with plating film electrode used to measure the body impedance, and the surface of the plating film electrode is larger than that of the capacitive electrode film. At the same time, this plating film electrode is connected to the port of the body impedance and water test circuit, the body impedance and water measurement circuit is connect to the two ports of the MCU system, one of which is the signal acquisition port of MCU systems, the other is a control port for controlling the body impedance and water test circuit. Weight sensor signal wire is connected with the weight signal processing circuit; and MCU system connected to weight signal processing circuit, display and keyboard by separated ports. The display and keyboard of the body composition monitor is set on the top surface of glass platform.

Compared with existing technology, the excellency of this invention is that, the test platform that made of insulating glass base with plating film electrode on it the glass stiffness is enough to bear the subject's weight so it does not need of other metal board construct, so the fee will be saving of pacific shell model, medal plat electrode model and producing.

### BRIEF DESCRIPTION OF THE FIGURES

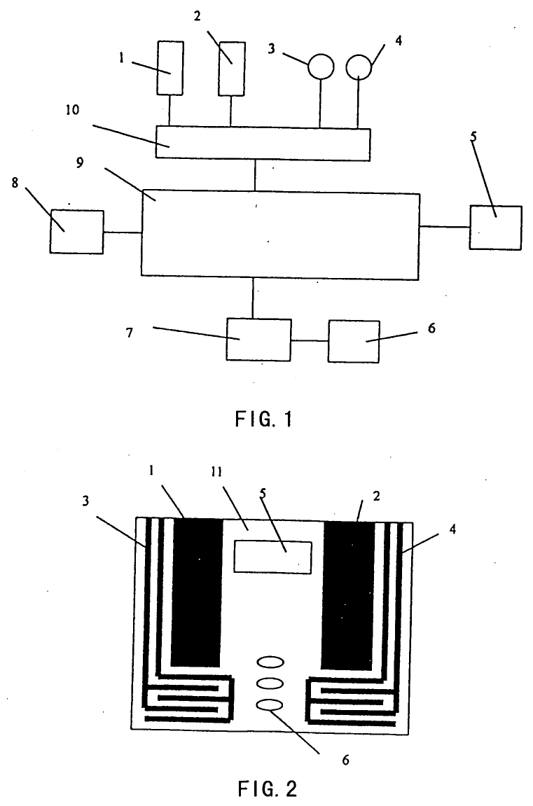

Referring now to FIG. 1, it shows a kind of schematic of this invention's system structure.

Referring now to FIG. 2, it shows a kind of structure schematic of glass test platform with plating film electrode.

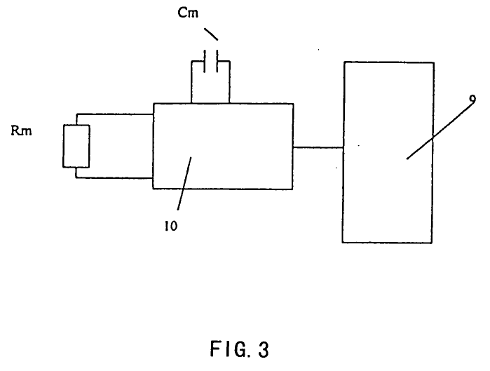

Referring now to FIG. 3, it shows a kind of test theory schematic of the body impedance and local subcutaneous tissue's permittivity.

### DETAILED DESCRIPTION OF THE PREFERRED EMBODIMENT

Refer to FIG. 1, on the glass test platform with plating membrane electrode, the plating film electrode 1 and 2 for measuring the body impedance and water liquid content circuit, and the capacitive film electrode 3 and 4, are connected to the port of the body impedance and water content circuit 10, the body impedance and water content circuit 10 is connected with two ports of MCU system 9, one of them is the signal acquisition port of MCU systems 9, the other is a control port for MCU system 9 sending a switch order to the body impedance and water measurement circuit 10, this order can switch the measure signal between the body impedance and local subcutaneous tissue's permittivity. The signal wire of the weighing sensor 6 connects with weighing signal processing circuit 7, the signal after processing enter one port of MCU system 9 by the weighing signal processing circuit 7. The display 5 connects with one port of MCU system 9, and the keyboard 8 connects with another port of MCU system 9.

The body impedance as mentioned above is an important parameter in the measuring the body fat content.

Refer to FIG. 2, on the top surface of the insulating glass test platform 11, there are two pieces of film electrode 12, this two electrodes are of proper surface area and are insulating to each other. And there are two groups of the capacitive electrode film 3 and 4 on the top surface of the glass test platform, each group include two pieces of electrode which is uniform space and non-touch to each other, the two pieces of electrode is insulating between each other, and the two groups of electrode are insulating from each other too. The display 5 and keyboard 6 is on the glass platform, and the surface acreage of film electrode 1 and 2 is bigger than that of the capacitance grid film.

The FIG. 3 is the test-philosophy schematic of this invention. The body impedance and water contend measurement circuit 10 is positive feedback RC oscillation circuit, through the contact between the subject's two soles and electrode 1 and 2, the body impedance Rna is connected with body impedance and water content measurement circuit 10. Through the contact between the subject's two soles and the capacitive electrode membrane 3 and 4, the subcutaneous capacity Cm is connected with body impedance and water content measurement circuit 10. The output terminal of body impedance and water content measurement circuit 10 is connected to the port of the MCU system 9.

The working process of this invention is: when a barefoot person stand on the glass test board 11, his two feet separately contact with the body impedance membrane electrode 1 and 2 and the capacitive electrode membrane 3 and 4 on the top surface of the glass test platform, through the output frequency signal of the body impedance and water liquid measurement circuit 10, which is correlative with the body impedance and the local subcutaneous permittivity, can get the body impedance and the local subcutaneous permittivity, at the same time, get the person's weight from the weighing sensor 6 and the weighing signal processing circuit 7. After MCU system 9 operate with the data that include the weight, body impedance, local subcutaneous permittivity and the person's sex, height, age, weight, body fat content and water content will be shown in the screen of the display 5.

1. A body composition monitor of glass platform plating film electrode comprising: the glass test platform that have plating film electrode which is used to measure body impedance on the surface, weight sensor and correlative circuit, MPU system, the body impedance and liquid content measurement electrode and the correlative circuit, display, keyboard;
On the top surface of glass test platform, there is at least one group capacitive electrode film used to measure the permittivity of subcutaneous tissue as well, one group of the electrode film has two piece of uniform piston and non-touch electrode film, which are non-conducting with each other and with other electrodes.

2. The body composition monitor as mentioned in the claim 1 has characteristics in:
The glass test platform that has plating film electrode used to measure body impedance on its top surface, the surface area of its plating film electrode is larger than that of capacitive film electrode.

3. The body composition monitor as mentioned in the claim 1 has characteristics in:
The glass test platform that has plating film electrode used to measure body impedance on its top surface, the plating film electrodes are connected to the ports of body impedance and liquid content measurement circuit, the body impedance and liquid content measurement circuit is connected to the two ports of MCU system, one port is the MCU system's signal acquisition port, the other is the MCU system's controlling port which control the body impedance and water content measurement circuit; weight sensor's signal wire is connected with the weight signal processing circuit; and MCU system is connected with the weight signal processing circuit, display and keyboard as well.

4. The body composition monitor as mentioned in the claim 1 has characteristics in:
The display screen and keyboard are located in the top surface of the glass test platform.
